(19) United States
(12) Patent Application Publication
(10) Pub. No.: US 2009/0266290 A1
(43) Pub. Date: Oct. 29, 2009

(54) Buoyant-capable beverage and food content-sensor

(75) Inventors: John W. Sliwa, Los Altos Hills, CA (US); Bryan T. Oronsky, Los Altos, CA (US); Herbert L. Berman, Palo Alto, CA (US); Neil C. Oronsky, Los Altos Hills, CA (US)

Correspondence Address: David W. Collins;Intellectual Property Law, Suite 100, 512 E. Whitehouse Canyon Road, Green Valley, AZ, 85614, US

(73) Assignees: Neil C. Oronsky; John W. Sliwa; Bryan T. Oronsky; Hebert L. Berman

(21) Appl. No.: 12/150,505

(22) Filed: Apr. 28, 2008

(51) Int. Cl. G01N 33/00

(52) U.S. Cl. 116/206

(57) ABSTRACT

A buoyant-capable sensor system device is disclosed for use alone or on an apparatus such as a straw, stirrer, mixer, stick or utensil which detects the presence or absence of one or more substances in a beverage or liquid food such as soup comprising a sensing element(s) which is either in direct or indirect contact with the beverage or food and a buoyant sensor body to assure user-visibility of the sensing element(s).

### BACKGROUND OF THE INVENTION

1. Field of the Invention

This invention relates to a portable and preferably disposable sensing device for detecting the presence of different chemical species or conditions in beverages or food items, and a method of using such an apparatus. The sensing device employs a buoyancy means for supporting its wettable sensing elements at or near the beverage or food-surface such that the sensing element can be visually read by the user.

2. Background Art

It would be particularly useful to be able to employ a simple, convenient, discreet and portable apparatus to detect the presence or absence of different substances in foods and solutions such as beverages since the consumer may not be aware of what he or she is actually ingesting. The apparatus of this invention advantageously enables the user to rapidly be able to determine whether a beverage being served contains (or does not contain) certain substances, impurities or adulterations therein. Representative examples of substances which may be checked for may include medications, drugs of abuse or “date rape” drugs, pollutants, toxins, poisons, bacteria, allergens, caffeine, glucose and lactose.

The prior art contains multiple examples of portable detectors to indicate the presence of certain substances or conditions in liquid materials.

For example, in U.S. Pat. No. 5,610,072, a dipstick is employed to measure the caffeine content of a beverage which is wicked to a reagent-impregnated section causing a color change to be observed.

An indicator is disclosed in WO 96/27795 in which caffeine and lactose are detected by wicking the beverage to a reagent which will react with the compounds to form colored products.

Various straws, sticks, mixers, and stirrers have been envisioned which employ similar methodologies to detect different compounds. Notably, none of the prior art examples teach a floatable device or mechanism. As a result, from a practical standpoint, we find that their beverage or food detection techniques suffer from a number of disadvantages and shortcomings.

First, the level of fluid in containers and the size of containers being variable, one size cannot fit all with the result that the prior-art dipstick, straw, mixer, toothpick, stirrer, stick or the like may be either too long or too short. Second, without the provision of buoyancy, these items can fall into the beverage through user carelessness or in case the bottom of the container is deeper than the length of the stick and remain submerged; this risks contamination of the liquid both from the chemicals on the sensor and from one's hands fishing the device out of the beverage. Moreover, the visual portion of the detector can be concealed or obscured by the fluid itself as it sloshes, pours or spills in or out of the container. Thirdly, whereas the prior art examples are not integratable or interfaceable with standard accessories in bars and restaurants, the present invention, preferably containing a central through-hole, clasp or the like is capturable, enabling the user to place it on any manner of straw, stirrer, mixer, stick or utensil in the environment and slide it to the desired height or depth for rapid, adaptable and unobtrusive monitoring. The shape of the device, if further desired, can be made to conform to any shape or design such as a slice of citrus or an ice cube to add to its unobtrusiveness. Finally, since a floating sensor is not required to be held by fingers for an extended period, this is a substantially “hands-free” device, enabling one to be able to drink his/her beverage safely in a clean way without being overly worried even if his/her hands are not clean.

### SUMMARY OF THE INVENTION

A visually readable sensor system is provided for detecting at least one of a presence of, absence of, or concentration of a species of interest or concern in a consumable beverage or food item. The sensor system comprises:

a sensor body which can be presented to or placed upon a surface of the beverage or food item and which is capable of at least one of floatably or buoyantly retaining, maintaining or recovering that location or a similar surface location;

a sensing material, composition or element capable of changing visual appearance in response to the detection of at least one of the presence, absence-of or concentration of the species in the beverage or food item;

the sensing material composition or element being situated one or more of upon, within, or coupled to the sensor body or comprising the sensor body;

a transport path for at least one food or beverage-related constituent or species to reach the sensor material from the beverage or food item;

the surface placement of the sensor system allowing for flow-communication, wetting or diffusive transport of at least one beverage or food constituent or species along the transport path to, upon or into the sensing material, composition or element; and

the sensing material, composition or element thereby changing an aspect of visual appearance in accordance with a state or degree of presence or absence of the species of interest in the beverage or food item.

A method of detecting a species of interest or concern in a beverage or food-item is also provided. The method comprises:

providing a buoyant capable species sensor capable of providing a visual indication of at least one of the specie's presence, absence or concentration in the beverage or food item while situated in a floated or buoyant state in or on the beverage or food item;

providing a beverage or food item which a user may or will ingest or may or will provide to another human or animal for their ingestion; and

causing the sensor to become situated at or in a surface of the beverage or food item whereat, after a period, it provides the visual indication to the user or ingester.

### BRIEF DESCRIPTION OF THE DRAWINGS

The sole FIGURE depicts, in section, an annular sensor device floating in a beverage with an optional straw or stirrer stick shown in phantom passed through or otherwise coupled to the sensor body.

### DETAILED DESCRIPTION OF THE EMBODIMENTS

A new type of buoyant-capable sensor used for detecting the presence or absence of different substances or conditions in foods and solutions such as beverages is taught herein. The sensor device is comprised primarily of a buoyant sensor body and a coupled or infused sensing element or material. In one embodiment, the device shape is that of a small ring, annulus, disc or puck a few to several millimeters in diameter. A ring-like or annular sensor easily accepts a straw or stirrer being passed through it such that it becomes captured to various degrees or in at least one degree of freedom by the straw or stick. In any case, the sensor either floats at the beverage surface or close enough to the surface despite possible sensor wetting such that its visual indicator can clearly be discerned. The sensor may also be mounted on an eating utensil and in some embodiments may even be adhered to a surface and utilized in a fixed position rather than a floating position. The sensing element may be wettable by the food item being tested; however, also within our inventive scope is the sensor detecting substances via vaporous, gaseous or solid-state diffusion. In another embodiment, the sensor may be prewetted as by a hydrogel so as to enhance diffusive substance uptake from even relatively dry food items such as smoked meats.

One application of this device may be in the management of diabetes. It is paramount for the diabetic patient to control his or her diet and specifically to monitor the amount of sugar that is consumed.

In general, diabetic patients should avoid the ingestion of an excessive amount of processed sugar. Processed sugar, of course, appears in many different foods, and in particular soft drinks or carbonated beverages, which are commonly served. The patient may not be aware that the food or drink contains sugar and this device will aid in that assessment. Two representative examples of enzymes used to test for glucose, which are well known in the art, include glucose oxidase and hexokinase.

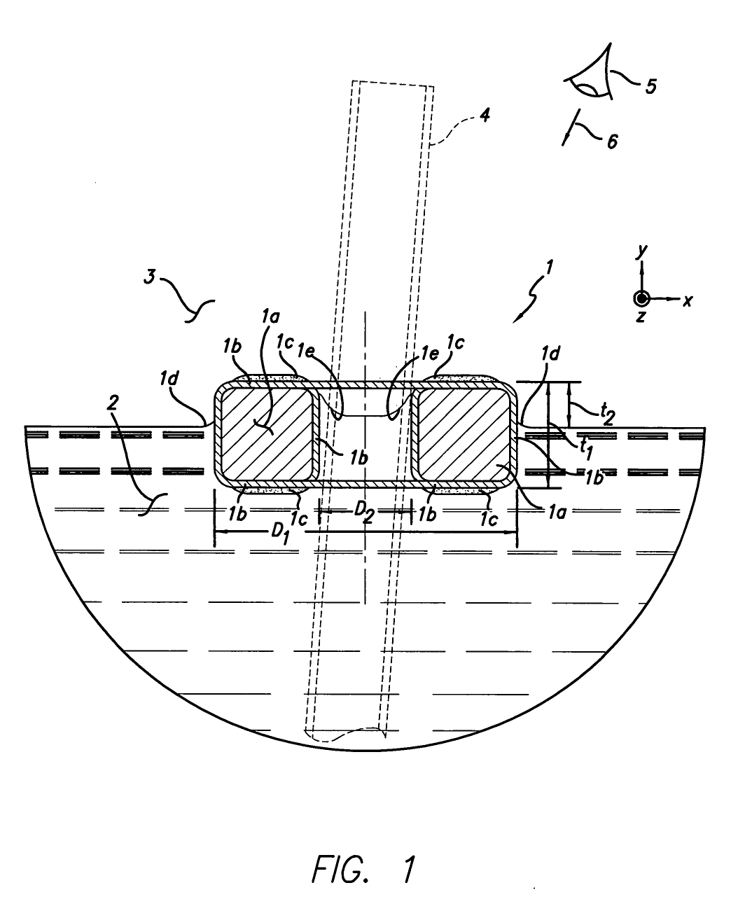

Moving now to FIG. 1, we see an inventive sensing device 1 in sectional view floating in a beverage 2 in an air ambient 3. The sensing device 1 is an annulus or ring as shown of outer diameter D1 and inner diameter D2 and thickness t1. The sensor 1 is shown captured around a straw or stirrer 4, shown in phantom, the sensor 1 being either fixedly captured or slidably captured thereon.

The sensor device 1 is comprised mainly of a sensor body material 1a that provides a structural platform for the additional components. Body material 1a is preferably buoyant in fluids such as beverages or sauces. Sensor body 1a is depicted as being coated with a permeable wicking material 1b, in this case depicted on all of its interior and exterior surfaces, although that is not required. Finally, a sensing material, composition or element 1c is depicted resident upon or infused into portions of the wicking material 1b. In the case shown, the sensing material 1c is situated on the top (+Y) and bottom (−Y) faces of the sensing device 1. We note that in FIG. 1 the sensing device 1 may function properly regardless of which side of the sensor 1 is oriented “up”.

It will be noted that a user or food-item consumer 5 may easily view along a line-of-sight 6 the state of the visual indicator or sensing material/element 1c and therefore determine if the substance or species of interest (or concern) is present, or how much of it is present.

In the depicted embodiment, the beverage or food material wets the wicking or absorptive coating 1b, and clear evidence of this are the meniscuses 1d on the diameter D1 and meniscuses 1e in the diameter D2. These meniscuses are comprised of wick-wetting beverage or food material. Such wetting may involve any of absorption, adsorption or capillary action.

Given the buoyancy of the sensor body 1a and the wetting of the wicking or absorptive/adsorptive layer or coating 1b, we can properly expect that the sensor device 1 will sit in or within the beverage surface. This is depicted with the dimension t2 which is the amount that the sensor remains sticking out of the beverage surface. Typically, t2 will be less than t1 but that is not a functional requirement.

It is critical to understand that by “buoyant” we mean that the sensor device 1 does not sink, or if sunk will return itself to or very near the beverage surface. This surface-returning propensity can be provided in at least two ways:
1) Use of a buoyant sensor body 1a or at least a net positive buoyancy of the overall sensor including all of its materials 1a, 1b, 1c and any wetted beverage (shown).
2) Use of surface-tension derived floatation forces such as those that allow insects to “walk on water”. Using this approach, we would make at least parts of the sensor device 1 unwettable (not shown).

By “returning to or near the surface” if sunk, we mean close enough to the surface such that sensor material 1c can be visually discerned by user 5. This does not require that the sensing material 1c remain dry. A thin film of beverage of flowable food material whose thickness is likely limited by surface tension can be easily seen through. In fact, even if the sensor device 1 is not pushed under the beverage surface its visible sensing material 1c will still be wetted with the help of the wicking layer 1b. Typically, the sensor material itself, item 1c, is also permeable.

In actual operation, in a first embodiment of use, the sensor device 1 would likely be unwrapped from a substantially hermetically sealed metalized wrapper which protects the sensor material 1c from moisture and/or light. The device 1 may then be dropped into or onto the food item or beverage, noting that as it lands in either orientation (+Y or −Y) a sensing face will be visible along viewing direction 6 (for the sensor of FIG. 1 with dual sensing faces). We note that many sensor shapes are possible, including those such as tetrahedrons and spheres. These shapes will always have visible faces when they are at or in the beverage surface.

Upon contact with the food item, in the wettable food scenario, the wicking coating 1b passes wettable food content up the wick (+Y direction), both on the inside and outside diameters D1 and D2. When it reaches the top sensor device surface, it will be also carried laterally (radially) across the top wick material 1b such that sensing material 1c is also exposed to it.

There are several design options and some of note are:
a) The sensing material may be coated upon and/or infused into the wick material.
b) The sensing material and the wicking material may be the same material.
c) The sensing material may be on some or all faces (or edges) of the device.
d) The sensing material may change color, shade, hue, or contrast/brightness, for example.
e) The overall shape of the sensor 1 is depicted as annular. It may also be spherical, rectangular, tetrahedral or cylindrical, for example.
f) The sensor body “material” la may be, for example, a foamed or porous material, thereby assuring buoyancy. It may be inherently bulk-unwettable or may be bulk or surface-treated to be unwettable.
g) The sensing coating material is preferably wettable and permeable but need not be as wettable as the wicking layer 1b if it is situated on such a wick. This is because the sensing material (on a wick) need not itself pump liquids more than through its own thickness.
h) The wicking material 1b may have more than one layer: an underlayer that seals the body material 1a and the permeable wettable overlayer.
i) In the case of a sensing device 1 having a hole (e.g., D2) in it for physical mating with a utensil such as the straw 4 shown in phantom, we stress that the hole D2 (or other capturing feature) may have compliant or deformable flaps or features providing a degree of fit-adjustability to different sized straws or sticks (compliant elements not shown). Ideally, these can be inactivated by the action of the straw being inserted if one wished the sensor device 1 to freely slide up and down the straw and/or buoyantly float on the straw on the beverage.

We note that any two or more of the body material 1a, the wicking material 1b, and the sensing material 1c may be the same material or altered or modified states of the same inherent material. In FIG. 1 we have depicted three separate materials.

Not shown in FIG. 1, but included in our inventive scope, is the sensor device 1 having an adhering ability such that it can be fixated to a convenient surface such as to the inside of a glass or cup, to a straw or stick or stirrer, to a spoon or fork or to a plate or bowl. Such an adhering ability could be provided with an adhesive member and the adhesive itself may contain the sensing material, particularly if only one adhesive face is adhered and the other is visible.

Thus, we have a sensing device which can be any of: floated on a food item surface, self-refloated on the food item surface if forcefully or accidentally sunk, slidably or non-slidably mounted on a straw or stick, or adhered to a food container or utensil.

A couple of sensor chemistries that may be used include the following:
1) For caffeine detection: using monoclonal antibodies reactive against caffeine.
2) For glucose detection: using hexokinase or glucose oxidase.

We anticipate the future use of our inventive sensor device to also detect pathogens, bacteria, viruses, spoiled food, spoiled wine (vinegar) and food adulteration/alteration as by sabotage, terrorism and biowarfare. To perform these functions, the sensor material or element may include a culturing or incubation capability and/or a genetically-engineered process. Given this, we stress that some applications, such as looking for sugar or so-called “date rape” drugs, will provide rapid answers in several seconds to a minute or so whereas applications looking for pathogens might take many minutes or hours. The invention does not require rapid readout; however, that is a desired behavior. For example, one could place the inventive sensors on food in the refrigerator and monitor that food for spoilage, a chemical or electrochemical signature of the spoilage being detected by the sensor. In that application, one would occasionally check the sensor over a period of days or longer, or check it upon each use of the stored food or beverage.

Some food items are relatively dry such as smoked meats. We therefore include in our inventive scope the sensor device 1 having a prewetted film such as a hydrogel whose purpose is to guarantee a diffusive wetted contact to the food item. The hydrogel (or wetting film/material) may or may not also contain the sensing material 1c but at least allows for some type of physical transport of various food species through the gel to the sensing material, such as by diffusion or ionic diffusion. Note that such transport might be primarily by diffusion within a relatively stagnant wetted film. Even this transport can occur on the order of a minute.

In other embodiments the sensor device 1 has additional user or ecologically beneficial features. Some of these embodiments may include, for example:
a) The sensor device may be biodegradable in the weather elements over a period if it is carelessly discarded as litter.
b) The sensor device, although not designed for ingestion, may be safely ingestable without medical complications.
c) The sensor device may be purposely arranged to be ingestable; i.e., it is itself a food item, decorative or otherwise.
d) The sensor devices may be provided or packaged in a dispenser which can easily dispense the desired number of devices, probably one per species, in a controlled manner from a protective packaged environment.
e) The sensor device may have visual indicators for two or more detectable species of interest, such as both for caffeine and sugar, or for two different drugs or medications.
f) By “visual” sensor we generally mean readable with the unaided eye. However, we include any manner of optical reading, including as by illumination or optical excitation with a penlight infrared or ultraviolet LED. This makes even more detection materials possible.
g) The sensor devices may be provided to the user or patron as by freezing them into ice cubes or ice cube surfaces. In this manner, a fresh drink with ice is provided having multiple freshly activated sensors.
h) One may additionally utilize the sensors in a game wherein the patron needs to collect a predefined set of them or a lucky one having a prize marker.
i) We have given specific examples of sensor materials and compositions above. We stress that, alternatively or additionally, the sensor may comprise a sensing element such as a micromechanical element utilizing MEMs technologies or nanoparticle technologies such as a nanoparticle-based sensing material. An attraction of a nanoparticle sensing material is that it makes it easy to have mixed nanoparticle types, each type of which detects its own species of concern. Using this approach, one can easily envision a sensor which can detect a dozen dangerous drugs. The visual indications for each may be identical or different. Nanoparticle approaches can also provide good wettability and sensitivity as needed.
j) We expect that these sensor devices will most likely be individually hermetically packaged and made available in restaurants in the manner of sweeteners or sugar-substitutes but in metalized hermetic packaging. In that application, they may also be provided with a mating straw or stick-separately or already preassembled.

It will be appreciated that if it is desired to minimize transfer of sensing material or its constituents into the beverage or food item, one may arrange for the food material (or the detectable species selectively) to have to pass through an intervening wick section or barrier before reaching the sensing material. For example, the device of FIG. 1 could be rearranged such that visually changeable sensing material is situated only above the beverage surface and beverage must be wicked upwards to it. Alternatively, the sensor device could be purposely momentarily immersed or stirred/swished around and then allowed to refloat. Upon refloating, the sensor material would be wetted but preferably have a long or non-existent continuous wetted path through a wicking path back to the beverage. Using this approach, there is minimal leaching of the sensor material into the food item, if any. Finally, one could coat the sensor material with a dissolvable coating which becomes wetted upon immersion but which dissolves into the wetted beverage film thereafter. In this manner, the sensor material is never itself appreciably exposed to the bulk beverage liquid.

Those familiar with microfluidics, capillary action, wetting, gels diffusion, and membrane exchange barriers will realize that several other variations of the invention are possible, such as versions which have osmotic exchange membranes overlying, underlying or comprising sensor materials. Such membranes may selectively only pass (or inhibit) species which are to be detected or have their presence assessed. More recently, color-changing gels have been reported; thus, such a gel may also be used such as the sensing material portion. Although we have taught embodiments of device sizes on the order of millimeters in diameter, we anticipate that one could make these devices very small. In that case, they may literally be poured onto or into food, particularly if they are edible.

The sensors may also be provided in a stick or plate batched or arrayed form wherein they are snapped off for individual use. Alternatively, they may be provided in clear blister-packs or bubble-packs as are many capsules and pills. Such packaging is protective of the sensors and allows the user to easily know how many he/she has remaining. One could fit a panel of them in one's wallet or purse.

Since the sensor optionally can be adhered to a surface for use, one may also place it on one's mucous membrane or tissue to detect drugs, medications, proteins, enzymes or pathogens for example. In this case, by “adhered” we simply mean that the device can be placed, for example, on the tongue or inner cheek and will not fall off unless physically dislodged as by the forceful tongue. This can easily be attained if the device is wettable, small, and preferably conforming. In that case, wetting surface tension itself can hold (adhere) it on the tissue long enough for sensing to take place. It will be recognized that such a device may be as thin as paper in order to provide the light weight and conforming behavior of some embodiments. The sensor device may even be given a flavoring to add a pleasant sensation and/or to mask the taste of a reagent which is not toxic in anticipated ingested quantities over time but tastes poorly.

By “wick” we mean any member or film which can pass or transport at least one constituent or species at least some distance along at least one dimension or direction. This is likely in a liquid state but we do not limit it to the liquid state. For example, surface diffusion can take place along or in films only a few molecules thick and such films are more properly referred to as adsorbate films or monolayer films rather than liquids. A “wick” may be volumetrically permeable or may have a wettable surface or surface film along/through which wetting can progressively take place. Such a wick may have macroscopic pores, microscopic pores or nanoscopic pores or intramolecule diffusion channels as would be expected of a nanomaterial or a hydrogel respectively. Wetting may even cause or be aided by a chemical reaction or electrochemical reaction with a constituent or species in the beverage or food material.

In use, at least one of the following may take place:
a) the user unpackages or dispenses at least one sensor and situates it in or on the beverage or food item whereupon it recovers or maintains a surface-visible position;
b) an item-preparer, item-manufacturer, item-packager, item-server, item-dispenser, item-seller, food preparer, chef, cook, waitress, server, bartender, stewardess or host places one or more sensors in a prepared or packaged beverage or food item and already in communicative contact with the item; or
c) an item-preparer, item-manufacturer, item-packager, item-server, item-dispenser, item-seller, food preparer, chef, cook, waitress, server, bartender, stewardess or host places one or more sensors together with a prepared or packaged beverage or food item, the user subsequently causing the sensor and the beverage or food item to contact each other.

In any event, any item-dispenser, server, manufacturer or seller may be being animate or inanimate.

What is claimed is:

1. A visually readable sensor system for detecting at least one of a presence of, absence of, or concentration of a species of interest or concern in a consumable beverage or food item comprising:
a sensor body which can be presented to or placed upon a surface of the beverage or food item and which is capable of at least one of floatably or buoyantly retaining, maintaining or recovering that location or a similar surface location;
a sensing material, composition or element capable of changing visual appearance in response to the detection of at least one of the presence, absence-of or concentration of the species in the beverage or food item;
the sensing material composition or element being situated one or more of upon, within, or coupled to the sensor body or comprising the sensor body;
a transport path for at least one food or beverage-related constituent or species to reach the sensor material from the beverage or food item;
said surface placement of the sensor system allowing for flow-communication, wetting or diffusive transport of at least one beverage or food constituent or species along the transport path to, upon or into the sensing material, composition or element; and
said sensing material, composition or element thereby changing an aspect of visual appearance in accordance with a state or degree of presence or absence of the species of interest in said beverage or food item.

2. The sensor system of claim 1 wherein the transport path includes or provides a path for any one or more of:
a) a wicking, absorbtive or adsorbtive material;
b) a diffusive path for an atom, molecule or ion;
c) a membrane;
d) direct transfer of at least one constituent or species from the beverage or food to, into or onto the sensor in at least one physical solid, liquid, gas, atomic, molecular, genetic or ionic state-such as by wetting or temporary immersion or submergence; or
e) any manner of electrochemical activity including current flow therefore.

3. The sensor system of claim 1 wherein the sensor system any one or more of:
a) can float, in which case it is resistant to permanent sinking or it resurfaces;
b) can float, in which case it has a portion that remains surface-exposed or surface-visible despite some amount of immersion and/or wetting;
c) can float, in which case it has a portion which remains close enough to a beverage or food surface such that the sensing material's visual appearance is visually detectable;
d) can float, in which case surface tension forces and/or buoyancy forces at least help keep it at or near the beverage or food surface or help return it to or near said surface; or
e) can be visually read although its readable region becomes wetted by the beverage or food item.

4. The sensor system of claim 3 wherein said floating is upright, in which case at least some visually readable sensing material, composition or element is upwardly facing or otherwise visible to the user as a result of the buoyancy tendency.

5. The sensor system of claim 1 wherein the sensing material, composition or element is any one or more of:
a) coated upon a sensor body surface;
b) infused into a sensor body surface;
c) infused into the bulk of the sensor body;
d) adhered to a sensor body;
e) fastened in any manner to a sensor body;
f) a reagent;
g) undergoes a chemical or electrochemical reaction;
h) is also the sensor body material or is a constituent thereof;
i) contains an enzyme;
j) contains a metabolite;
k) contains antibodies;
l) contains a hydrogel, wetting agent or other surfactant; or
m) is wettable or permeable or acts to wick or absorb one or more constituents or species.
n) is transport coupled to a transport path

6. The sensor system of claim 1 wherein the sensor system any one or more of:
a) can be slidably mounted upon any one or more of a drinking straw, swizzle stick, utensil or stirrer;
b) can be floated upon or at the surface of a beverage or food item;
c) can be immersed in a beverage or food item whereupon it substantially floats back to the surface or near to the surface to a readable position;
d) is dispensed from a dispenser of multiple such sensor systems;
e) allows for stirring of the beverage yet reasserts its surface or near-surface visible position;
f) has a central through-hole with flaps, clasp, adhesive, bendable wire or a shape-conforming aperture allowing for adjustable placement on or onto one or more of straws, stirrers, mixers, sticks and utensils;
g) is provided in a bubble-pack or blister-pack;
h) is provided in substantially waterproof packaging;
i) is provided in a metalized package; or
j) is provided with an adhesive, adhering or retainment member which allows for substantially static optional physical attachment to a solid surface.

7. The sensor system of claim 1 wherein the species being detected is any one or more of:
a) caffeine, using monoclonal antibodies reactive against caffeine; b) glucose, using hexokinase or glucose oxidase; c) a beneficial drug; d) a harmful drug; e) sugar; f) a poisonous or toxic substance; g) an addictive drug; h) an illegal drug; i) a species to which the consumer is or may be allergic; j) a carcinogen; k) a bacteria, spore or fungus; l) a virus; m) an alcohol; n) any undesired contaminant; o) any species whose measured presence has medical significance for the user; p) lead; or q) a heavy metal.

8. The sensor system of claim 1 wherein any of:
a) any portion of a sensor device is buoyant or unwettable by the beverage or food item, thereby contributing to sensor system buoyancy in or upon the beverage or food item;
b) any portion of a sensor device is rendered wettable or unwettable for any reason to a beverage or food item or constituent or species thereof;
c) any portion of a sensor device is porous, permeable or semi-permeable;
d) any portion of a sensor device is absorptive, adsorbtive or wicking;
e) any portion of a sensor device is coated or covered with an overlayer for any purpose;
f) any portion of a sensor device contains hyperosmolar or other osmotically active substances;
g) any portion of a sensor device contains a color chart or color indicator useful to interpret a sensor reading;
h) any portion of a sensor device is imprinted with a lot number or tracking number related to the sensor itself or to a related food or beverage item; or
i) any portion of the sensor device contains a hydrogel or other wetting-agent which supports species transport or detection.

9. The sensor system of claim 1 wherein the sensing material, composition or element is/are any one or more of:
a) arranged to be porous, permeable or semi-permeable to a beverage or food, to a constituent thereof, or to a species of interest or concern;
b) arranged to be wettable, absorbtive, adsorbtive, unwettable, nonabsorbing or nonadsorbing to a beverage or food, constituent thereof, or to a species of interest or concern;
c) situated, at least in part, on or in an upwards-facing or otherwise visible exposed surface of the sensor system during use;
d) situated, at least in part, on a beverage or food wetted or contacting surface or region of the sensor system during use;
e) capable of wicking or absorbing a beverage or food constituent or species during use;
f) is color or hue changing;
g) can change in a degree of lightness or darkness; or
h) can change visual behavior under at least one type of optical illumination.

10. The sensor system of claim 1 wherein the sensor system itself can be safely ingested, if ingested, at least once.

11. The sensor system of claim 1 wherein the sensor system is substantially hermetically sealed, including in a wrapping or blister pack, before use.

12. The sensor system of claim 1 wherein one or more surface tensions of the sensor system and/or the beverage or food item provides some or all of the sensor system's tendency to remain at or near said surface such that it is visually readable by the user.

13. The sensor system of claim 1 wherein two or more species can be detected by two or more sensing materials, compositions or elements, whether sequentially or simultaneously, the two or more sensing materials, compositions or elements being mixed or being separate.

14. The sensing system of claim 1 wherein the detected species or species of interest or concern relates to at least one of:
a) whether a beverage or food item is a diet or non-diet version;
b) whether a beverage or food item does or does not contain sugar;
c) whether the beverage or food item has been adulterated or spiked, including criminally;
d) whether the beverage or food item is caffeinated or not;
e) whether the beverage or food item may cause an allergic reaction;
f) whether the beverage or food item is spoiled or contaminated;
g) whether the beverage of food item contains fat or a fat related species;
h) whether an alcoholic drink has been unfairly diluted or is too rich in alcohol;
i) whether a non-alcoholic drink contains alcohol;
j) whether lactose is present or absent;
k) whether a toxin or poison is present or absent;
l) whether a microbial species is present such as a bacteria;
m) whether a mineral, vitamin or herb is present or absent; or
n) whether an allergen or any substance capable of inducing anaphylaxis is present.

15. The sensing system of claim 1 wherein also provided is a visual indicator or color chart such that the user understands what the visual states of the sensing material mean, the indicator or color chart being any one or more of: a) provided on the sensor itself, b) provided on a straw, stirrer, stick or other utensil used with the sensor, c) provided on the sensor packaging, d) provided on printed or electronically conveyed instructions for use, e) posted in a public place viewable, including by a diner, patron, shopper or customer, or f) provided together with the sensor packaging

16. The sensing system of claim 1 wherein the sensor system is exposed to the beverage or food item by at least one of:
a) said placement at or upon the surface of the beverage or food item;
b) immersion in the item followed by the sensor system returning to the beverage or food item's surface of its own accord, including as caused by buoyancy and/or surface tension forces;
c) placement of the sensor system in contact with a sample of the beverage or food item, the sample having been removed from a larger parent body of said beverage or food item; or
d) immersion in the beverage or food item and physical removal therefrom, the sensor system remaining wetted by the beverage or food item or at least having captured some amount of the item.

17. The sensing system of claim 1 wherein any of:
a) the sensor system is placed in contact with the beverage or food item by the user;
b) the sensor system is provided to the user already present in or on the beverage or food item, including as during beverage or food preparation, beverage or food manufacturing/packaging, beverage or food-dispensing or beverage or food-serving; or
c) the user or item-consumer is a human or animal.

18. The sensor system of claim 1 wherein the sensing material, composition or element utilizes any one or more of: a) genetic engineering, b) the culturing of pathogens, c) any chromagen, d) a chemical reaction, e) an electrochemical reaction, f) a reagent, or g) a fluorescent or phosphorescent label viewable under controlled illumination or excitation.

19. A method of detecting a species of interest or concern in a beverage or food-item, comprising:
providing a buoyant capable species sensor capable of providing a visual indication of at least one of the specie's presence, absence or concentration in the beverage or food item while situated in a floated or buoyant state in or on the beverage or food item;
providing a beverage or food item which a user may or will ingest or may or will provide to another human or animal for their ingestion; and
causing the sensor to become situated at or in a surface of the beverage or food item whereat, after a period, it provides the visual indication to the user or ingester.

20. The method of claim 19 wherein at least one of:
a) the user unpackages or dispenses at least one sensor and situates it in or on the beverage or food item whereupon it recovers or maintains a surface-visible position;
b) an item-preparer, item-manufacturer, item-packager, item-server, item-dispenser, item-seller, food preparer, chef, cook, waitress, server, bartender, stewardess or host places one or more sensors in a prepared or packaged beverage or food item and already in communicative contact with the item; or
c) an item-preparer, item-manufacturer, item-packager, item-server, item-dispenser, item-seller, food preparer, chef, cook, waitress, server, bartender, stewardess or host places one or more sensors together with a prepared or packaged beverage or food item, the user subsequently causing the sensor and the beverage or food item to contact each other.

21. The method of claim 19 wherein the buoyant capable sensor any of:
a) is captured in at last one degree of freedom by a straw, stick, stirrer, utensil or ice-cube;
b) is adhered or suctioned to a beverage or food container;
c) is adhered or suctioned to a mucous tissue or skin; or
d) receives a sub-sample of the beverage or food item taken from a larger parent body of said beverage or food item in any manner.

22. The method of claim 19 wherein one or more sensors are also utilized to play a game, the game utilizing a visual or readable attribute of the sensor at least one of before or after its sensing use, the game involving at least one sensor user or possessor thereof.

23. The method of claim 19 wherein the sensor, over a period of any one or more of minutes, hours, days, weeks or months, provides information relating to at least one of the items: a) spoilage state, b) freshness state, or c) a state of ripeness.

24. The method of claim 19 wherein the sensor is employed, at least in part, to assess the truthfulness of an organic labeling or organic description of the item.

25. The method of claim 19 wherein the beverage or food item is or contains, whether by accident or purposeful intent, any one or more of a: a) medicament, b) drug, c) vitamin or nutrient, d) mineral, e) a toxic material, f) a biowarfare agent, or g) a pathogen and the sensor is employed to detect at least one state of presence, absence or degree of concentration thereof.

26. The method of claim 19 wherein the sensor is employed to at least one of:
a) monitor spoilage or ripeness of beverage or food being stored or sold;
b) monitor adulteration of beverage or food being stored or sold; or
c) support an enforcement or monitoring action for any one or more civil, municipal, city, town, state, county, corporate, company or federal rule or regulation relating to beverage or food preparation, packaging, storage, sale, display, advertising, labeling, safe-use, truth-in-advertising, or consumption.

27. The method of claim 19 wherein the sensor or its packaging bears a lot number, date-code, customer-information or identifying-code, thereby allowing a user or customer buying or employing the sensor(s) to determine any one or more of:
a) the particular sensor's continued viability in view of a period of time which has passed since sensor manufacture;
b) the species which the particular sensor can sense or detect;
c) the particular sensor's sensitivity to one or more sensed species;
d) the particular sensor's ability to detect a designer drug; or
e) the particular sensor's ability to detect a class of drugs.
